(19) United States
(12) Patent Application Publication
(10) Pub. No.: US 2005/0186226 A9
(43) Pub. Date: Aug. 25, 2005

(54) Method of treating diseases connected with immunity deficiency

(76) Inventor: Yakov Yevseyevich Brilman, Voorhees, NJ (US)

Correspondence Address: YAKOV BRILMAN, 219 Laurel Rd, apt. 508, Voorhees, NJ, 08043, US

(21) Appl. No.: 10/421,660

(22) Filed: Apr. 23, 2003

(51) Int. Cl. A61K 39/02; C12N 1/20

(52) U.S. Cl. 424/234.1; 435/252.3

(57) ABSTRACT

The invention is intended for treatment of diseases in accordance with immunity deficiency (HIV/AIDS diseases, oncological ailments and other). Purpose of it is reinforcement immunological reactivity organism. The method autohemovaccineimmunotherapy (hemovaccineimmunotherapy), diversified from prototype that that hemolyzed blood from sick person and inactivated at 56° C. for 30-40 min. is used with following accompaniment to its noinactivated serum blood from healthy animal unreceptive to the stirred of disease (mixture “A”) and serum blood from healthy person (necessary group and RH-factor) (mixture “B”) and 5% solution of the sodium cloride in necessary correlations. Partial destruction of the antigen (forming the autovaccine) is reached by the thermal influence on the hemolyzed blood of sick person with the same time destruction of the broken properdini system. The addition of other serum blood brings the appearance of new immune reagent. All variety of parenteral enters are used in this method. Subdermal and intradermal injections are conducted in some active points of the skin.

The invention pertains to the medicine and is intended for treatment of diseases in accordance with immunity deficiencies.

The method of the treatment with a vaccine what is prepared from microbe chosen from a sick person (autovaccine) is known.

The method of the treatment by their own blood—autohemotherapy also is known. It consists of extraction from the vein of a sick person and subsequent injecting of (the blood) into a muscle or under the skin.

Stimulating effect of autohemotherapy is explained with the impact of albumin-like products of the dissociation of blood protein, freeing in the process of splitting by enzymes under the influence of protease. These dissociation products are irritants of various systems of a sick organism.

The research of A. A. Bogomolets and his learners showed that the products of dissociation of eritrocytes were activators of the function of reticuliendotelium system. In order to activate this function they suggested the treatment with own hemolyzed blood.

The latter method was applied for treating acute inflammatory and abscesses, for peptic ulcer, etc.

It is known that the infusion of the very small amount of the blood of a different group, acting mainly on defensive systems of organism: i.e. reticuloendotheliosis and blood creating systems, activates the organism protective forces and strengthens the vital functions of tissues, which are suppressed predominantly with lingering intoxications and infections.

However, the autohemotherapy according to the technique described above is not effective enough and is rarely used in therapeutic practice.

It is well-known that immunological reactiveness of the organism includes the fluid internal environs of the organism (humoral) and cell immunity, cell functions of immunological memory (T- and B-Lymphocytes) and presence of immune components/the system of properdini (one of the mechanisms natural immunity), which consists of complementum of properdini and salts of magnesium, antibodies, interferoni, etc./.

The appearance of “imbalance” of any of component of this complex intercommunicated immunological system makes improvements in immunological processes of the organism.

Examples of it can be tolerance of the organism on the malignant formations, weak immunological reactiveness during some of the chronic diseases, immunological processes during the rheumatoid arthritis, systemic lupus, etc.

There are some well-known ways of active and passive immunization.

First is an injection of antigens. A more broad generalized form of active immunization is a vaccination.

Passive immunization is done by an injection of serum or serum's fraction of blood of immune animals and people under the skin, or by intramuscular or intravenous injection. This preparation includes prepared antibodies which neutralize toxin, inactivate pathogens and prevents its spreading.

During my investigation I found that the serum of blood of people who have malignant tumour, or who have 30 day long sickness or more, because of viral hepatitis which doesn't react to one of the reaction antigen-antibody. They begin to react only after it inactivate of thermal unstable components of immune system of its serum during the temperature of 56° C. during 30 min. with adding of some drops of normal serum of blood of healthy person.

Coming is thereof offered method of the influence on immune system of the organism and treatment of the row of the sicknesses (HIV/AIDS disease, tuberculosis, oncological ailments, chronic pneumonia, rheumatics, chronic bronchitis, etc.).

This offer has drawn confirmation in one experiment. Thus, during the flashing of pasteurellosus among a group of rabbits, of them that had this kind of disease. One timed injection (subdermal) of mixture (inactivated haemolyzed blood sick rabbits with accompaniment several drops serum blood from person) gave a big improvement of their health. Experimental animals become more active, the appetite becomes better, they became healthy very quickly. None of them died. Nevertheless, the change for the worse among non-cured rabbits was evident and the mass rabbit died.

Therefore I am offering a complex method for treating diseases connected with immunity deficiency. The medical composition is found in base of the method, capable of intensify immunity reactivity organism as way of autovaccination so and by immunological influence components proposed composition on the immunity system of the organism. With some purpose it in combination with different type of the introduction mixture in organism—subdermal, intradermal, intramuscular, and intravenous, and also adviseable characteristic the homolyzed blood is also used.

The proposed method of the treatment presents a method of autohaemovaccinoimmunotherapy (or vaccinoimmunohaemotherapy) diversified from the prototype (autohemotherapy with hemolyzed own blood) that hemolyzed blood is used first, for reception an autovaccine. Under hemolyzed blood alongside with destruction of eritrocytes, leucocytes as well as destruction, as a result a virus will turn out to be outside of leucocyte, and will be easier to undergo for after-treatment, which is necessary. Second this hemolyses blood subject to thermal processing under 56° C. 30-40 min. to achieve autovaccine consisting of antigen-virus lest activity (for active immunization). Third, such thermal process of the blood brings to destruction the thermal unstable components of the broken properdine system. Following accompaniment to this inactivated hemolyzed blood non-inactivated serum blood of a sound animal possessing natural immunity to this infection with following also using serum blood of a sound person corresponding to groups and RH-factor, cause chain reaction on normalization broken properdine system in organism and creates a push increasing the immune answer to antigen.

Besides introduction to organism alien protein in small amount agitates defensive power of the organism.

The proposed method also differs from prototype of my application for invention for “Medical Composition” registered in “ [symbol] ” (Moscow) in 1982 y. N3526124/14 (162540) * that in offered method of the treatment use in addition a mixture “A” (see footnote below) and complex injection of these mixtures in organism. Purpose of the proposed method of the treatment is a making the medical composition, capable to intensify immunological reactivity of organism. This purpose is reached by that, that in medical composition which consists of blood from a sick person, use in activated hemolyzed blood of sick person in mixture with noninactivated serum blood from healthy unreceptive to the disease animal and also noninactivated serum blood from healthy person.
*I did not get a patent for this invention (“Medical Composition”) because the approbation was never done for some reasons.

This mixtures “A” and “B” in weight % is prepared:

Mixture “A”
Blood from sick person—60
Sterile bidistilled water—30
Serum blood necessary healthy animal—5
5% solutions NaCl—5

Under conditions of strong aseptic the needed amount of blood is taken. Then it is haemolyzed by taking it of sick into sterile syringe. The syringe must contain a volume of blood and the half volume sterile bidistilled water.

When the haemolysis is completed the blood is inactivated in a inactivator under 56° C. 30-40 min. After the inactivated haemolyzed blood has cooled down, add necessary amount noninactivated serum from healthy animal immune to the infection and 0.5 ml. sterile 5% NaCl solution. The mixture enter on scheme (1-2 days).

Mixture “B”—prepare also as mixture “A” but instead of noninactivated serum from animal use serum blood from healty person,

Consistency:
Blood from sick person—56-60
Sterile bidistilled water—28-30
Serum blood from sound person (necessary group and RH-factor)—5-10
5% NaCl solution—5-6
   Herewith use all types injections in organism: intradermal, subdermal, intramuscular and intravenous.

Schemes of the treatment can be different for that or other diseases.

Example of the scheme of the treatment of the AIDS disease is offered.

The mixture “A” prepare at first day of the treatment: to 5.0 ml. blood from a sick person add 2.5 ml. sterile bidistilled water mix it. After that this haemolyzed blood is inactivated in an inactivator at 56° C. for 40-45 min. After the inactivated haemolyzed blood mixture has cooled down, add 0.5 ml. of noninactivated serum blood from animal immune to the infection (possible ram or other animal) and 0.5 ml. sterile 5% NaCl solution. This mixture is given in one-shot to the same sick person whose blood was taken: intramuscular 3.0 ml. in each buttock, subcutaneus—1.0 ml. and intracutaneus (from surface forearms) on 0.1 ml. (whole 0.2 ml.).

In day following first procedure this mixture enter sick again only intradermal in the internal surface forearms on 0.1 ml.

In day following second procedure prepare mixture “B”. Beside sick person take from vein 7.0 ml. blood and mixed with 3.5 ml. sterile bidistilled water. After haemolysis this liquid row in inactivator under 56° C. during 30-40 min. and then to it add 1.0 ml. serum blood from healthy person (necessary group and RH-factor), mix and also add 0.5 ml. sterile 5% solutions of the sodium cloride. This mixture enter the sick person in same volume and also as it is described for first day treatment with accompaniment intravenous injection 3.0 ml. mixture “B”. The following day treatments this medical composition (mixture “B”) enter only intracutaneus in the internal of the surfaces forearm in both hands alternately on 0.1 ml. each day, enlarging daily amount of the places of the introduction on one and bring them before 10 injections.

Complex injections the mixture “B” in organism conduct as it is described for second day procedures in day 5-6 once, but in gap between they conduct only intradermal introduction as it is described above. With provision for possible insufficient presence in organism of the incitant infections for reception efficient medical autovaccine beside persons fresh infected (before 2-h˜3-h week after poisoning) by diseases connected with immunity deficiency (HIV-infection and others) is offered instead use inactivated and haemolyzed own blood infected person use in above described medical mixtures “A” and “B” inactivated and haemolyzed blood from person, infected the patient (best variant) or such blood from sick sufferring sister variety of the stirrer of this disease. Herewith from above described course injection with use of this vaccine for preventive maintenance and early treatment above specified diseases, will exclude intravenous ad intramuscularis injections.

The proposed method of the treatment has no contraindications.

The treatment can be conducted in combination with common methods.

### ANNOTATION

The invention pertains to part of medicine and is intended for treatment of diseases in accordance with immumity deficiency (HIV/AIDS diseases, oncological ailments and other). Purpose of the invention is reinforcement immunological reactivity organism. To this effect offered use a method autohaemovaccineimmunotherapy (haemovaccineimmunotherapy), diversified from prototype following:

1. Haemolysed blood from sick person and inactivated at 56° C. for 30-40 min. use with following accompaniment to its noinactivated serum blood from healthy animal unreceptive to the stirred of disease (mixture “A”) and serum blood from healthy person (necessary group and RH-factor) (mixture “B”) and 5% solution of the sodium cloride in below following correlations:

Mixture “A”—
blood from sick person—60
sterile bidistilled water—30
serum blood from necessary sound animal—5
5% solution of the sodium cloride—5

Mixture “B”:—
Blood from sick person—56-60
Sterile bidistilled water—28-30
Serum blood from sound person—5-10
5% solution of the sodium cloride—5-6

2. For the reason preventive maintenance and early treatment of the diseases in accordance with immunity deficiency (HIV-infection and other) beside persons fresh infected (before 2-3 weeks after contamination) it is offered conduct course an injection mixtures “A” and “B”, in which instead of haemolyzed and inactivated own blood from the infected person use haemolyzed and inactivated blood or person of the infected patient, or other sick with similar disease (infected such type of the infection).

In the last case excluded from course injection intravenous and intramuscular introductions.

3. Method complex injections is used to organism sick person mixtures “A” and “B” way intradermal, subdermal, intravenous and intramuscular injections on scheme.

1. What I claim as my invention is: Method of treating diseases connected with immunity deficiency such as HIV/AIDS disease, oncological ailments, tuberculosis, rheumatics, etc. by autohaemovaccinoimmunotherapy.
